(19) United States
(12) Patent Application Publication
(10) Pub. No.: US 2023/0399373 A1
(43) Pub. Date: Dec. 14, 2023

(54) MOLECULAR DESIGNS OF GLUCOSE-RESPONSIVE AND GLUCOSE-CLEAVABLE INSULIN ANALOGUES

(71) Applicant: THE TRUSTEES OF INDIANA UNIVERSITY, Bloomington, IN (US)

(72) Inventors: Michael A. WEISS, Indianapolis, IN (US); Balamurugan DHAYALAN, Carmel, IN (US); Mark A. JAROSINSKI, Fishers, IN (US)

(21) Appl. No.: 18/033,229

(22) PCT Filed: Oct. 22, 2021
(86) PCT No.: PCT/US21/56215

§ 371 Date: Apr. 21, 2023

Related U.S. Application Data
(60) Provisional application No. 63/104,196, filed on Oct. 22, 2020.

(51) Int. Cl. C07K 14/62

(52) U.S. Cl. CPC C07K 14/62; A61K 38/00

(57) ABSTRACT

A two-chain insulin analogue is provided containing (a) a B chain modified by a C-terminal diol element such that one hydroxyl group substitutes for the C-terminal carboxylate function in combination with (b) a glucose-binding element attached to the A chain at or near its N terminus. Compositions comprising such insulin analogs are used in methods of treating a patient with diabetes mellitus.

### CROSS REFERENCE TO RELATED APPLICATIONS

This application is a U.S. national counterpart application of international application serial NO. PCT/US2021/056215 filed Oct. 22, 2021, which claims priority to U.S. Provisional Patent Application No. 63/104,196 filed on Oct. 22, 2020, the disclosures of which are hereby expressly incorporated herein by reference in their entireties.

### INCORPORATION BY REFERENCE OF MATERIAL SUBMITTED ELECTRONICALLY

Incorporated by reference in its entirety is a computer-readable nucleotide/amino-acid sequence listing submitted concurrently herewith and identified as follows: 15 kilobytes ACII (text) file named “348446_ST25.txt,” created on Oct. 22, 2021.

### BACKGROUND

The engineering of non-standard proteins, including therapeutic agents and vaccines, may have broad medical and societal benefits. Naturally occurring proteins—as encoded in the genomes of human beings, other mammals, vertebrate organisms, invertebrate organisms, or eukaryotic cells in general—may have evolved to function optimally within a cellular context but may be suboptimal for therapeutic applications. Analogues of such proteins may exhibit improved biophysical, biochemical, or biological properties. A benefit of protein analogues would be to achieve enhanced activity (such as metabolic regulation of metabolism leading to reduction in blood-glucose concentration under conditions of hyperglycemia) with decreased unfavorable effects (such as induction of hypoglycemia or its exacerbation).

An example of a therapeutic protein is provided by insulin. Wild-type human insulin and insulin molecules encoded in the genomes of other mammals bind to insulin receptors in multiple organs and diverse types of cells, irrespective of the receptor isoform generated by alternative modes of RNA splicing or by alternative patterns of post-translational glycosylation. An example of a medical benefit would be the non-standard design of a soluble insulin analogue whose intrinsic affinity for insulin receptors on the surface of target cells, and hence whose biological potency, would depend on the concentration of glucose in the blood stream. Such an analogue may have a three-dimensional conformation that changes as a function of glucose concentration and/or may have a covalent bond to an inhibitory molecular entity that is detached at high glucose concentrations. Although it is not presently known in the art how to engineer such hypothetical analogues, this long-sought class of protein analogues or protein derivatives is collectively designated “glucose-responsive insulins” (GRIs).

The insulin molecule contains two chains, an A chain, containing 21 residues, and a B chain containing 30 residues. The mature hormone is derived from a longer single-chain precursor, designated proinsulin, as outlined in FIG. 1. Specific residues in the insulin molecule are indicated by the amino-acid type (typically in standard three-letter code; e.g., Lys and Ala indicate Lysine and Alanine) and in superscript the chain (A or B) and position in that chain. For example, Alanine at position 14 of the B chain of human insulin is indicated by Ala^{B14}; and likewise Lysine at position B28 of insulin lispro (the active component of Humalog®; Eli Lilly and Co.) is indicated by Lys^{B28}. Although the hormone is stored in the pancreatic β-cell as a Zn²⁺-stabilized hexamer, it functions as a Zn²⁺-free monomer in the bloodstream. The three-dimensional structure of an insulin monomer is shown as a ribbon model in FIG. 2. Pertinent to the logic of the present invention is the proximity of the C terminus of the B chain (B30) to the N terminus of the A chain (A1), often engaged in a salt bridge (FIG. 3A). Covalent tethering of these terminal ends blocks binding of the hormone analogue to the insulin receptor (FIG. 3B) as such a tether blocks a conformational switch on receptor engagement.

Administration of insulin has long been established as a treatment for diabetes mellitus. A major goal of conventional insulin replacement therapy in patients with diabetes mellitus is tight control of the blood glucose concentration to prevent its excursion above or below the normal range characteristic of healthy human subjects. Excursions above the normal range are associated with increased long-term risk of microvascular disease, including retinopathy, blindness, and renal failure. Hypoglycemia in patients with diabetes mellitus is a frequent complication of insulin replacement therapy and when severe can lead to significant morbidity (including altered mental status, loss of consciousness, seizures, and death). Indeed, fear of such complications poses a major barrier to efforts by patients (and physicians) to obtain rigorous control of blood glucose concentrations (i.e., exclusions within or just above the normal range), and in patients with long-established Type 2 diabetes mellitus such efforts (“tight control”) may lead to increased mortality. In addition to the above consequences of severe hypoglycemia (designated neuroglycopenic effects), mild hypoglycemia may activate counter-regulatory mechanisms, including over-activation of the sympathetic nervous system leading to turn to anxiety and tremulousness (symptoms designated adrenergic). Patients with diabetes mellitus may not exhibit such warning signs, however, a condition known as hypoglycemic unawareness. The absence of symptoms of mild hypoglycemia increases the risk of major hypoglycemia and its associated morbidity and mortality.

Multiple and recurrent episodes of hypoglycemia are also associated with chronic cognitive decline, a proposed mechanism underlying the increased prevalence of dementia in patients with long-standing diabetes mellitus. There is therefore an urgent need for new diabetes treatment technologies that would reduce the risk of hypoglycemia while preventing upward excursions in blood-glucose concentration above the normal range.

Diverse technologies have been developed in an effort to mitigate the threat of hypoglycemia in patients treated with insulin. Foundational to all such efforts is education of the patient (and also members of his or her family) regarding the symptoms of hypoglycemia and following the recognition of such symptoms, the urgency of the need to ingest a food or liquid rich in glucose, sucrose, or other rapidly digested form of carbohydrate; an example is provided by orange juice supplemented with sucrose (cane sugar). This baseline approach has been extended by the development of specific diabetes-oriented products, such as squeezable tubes containing an emulsion containing glucose in a form that can be rapidly absorbed through the mucous membranes of the mouth, throat, stomach, and small intestine. Preparations of the counter-regulatory hormone glucagon, provided as a powder, have likewise been developed in a form amenable to rapid dissolution and subcutaneous injection as an emergency treatment of severe hypoglycemia. Insulin pumps have been linked to a continuous glucose monitor such that subcutaneous injection of insulin is halted and an alarm is sounded when hypoglycemic readings of the interstitial glucose concentration are encountered. Such a device-based approach has led to the experimental testing of closed-loop systems in which the pump and monitor are combined with a computer-based algorithm as an “artificial pancreas.”

For more than three decades, there has been interest in the development of glucose-responsive materials for co-administration with an insulin analogue or modified insulin molecule such that the rate of release of the hormone from the subcutaneous depot depends on the interstitial glucose concentration. Such systems in general contain a glucose-responsive polymer, gel or other encapsulation material; and may also require a derivative of insulin containing a modification that enables binding of the hormone to the above material. An increase in the ambient concentration of glucose in the interstitial fluid at the site of subcutaneous injection may displace the bound insulin or insulin derivative either by competitive displacement of the hormone or by physical-chemical changes in the properties of the polymer, gel or other encapsulation material. The goal of such systems is to provide an intrinsic autoregulation feature to the encapsulated or gel-coated subcutaneous depot such that the risk of hypoglycemia is mitigated through delayed release of insulin when the ambient concentration of glucose is within or below the normal range. To date, no such glucose-responsive systems are in clinical use.

A recent technology exploits the structure of a modified insulin molecule, optionally in conjunction with a carrier molecule such that the complex between the modified insulin molecule and the carrier is soluble and may enter into the bloodstream. This concept differs from glucose-responsive depots in which the polymer, gel or other encapsulation material remains in the subcutaneous depot as the free hormone enters into the bloodstream. An embodiment of this approach is known in the art wherein the A chain is modified at or near its N-terminus (utilizing the α-amino group of residue A1 or via the ε-amino group of a Lysine substituted at positions A2, A3, A4 or A5) to contain an “affinity ligand” (defined as a saccharide moiety or diol-containing moiety), the B chain is modified at its or near N-terminus (utilizing the α-amino group of residue B1 or via the ε-amino group of a Lysine substituted at positions B2, B3, B4 or B5) to contain a “monovalent glucose-binding agent.” In this description the large size of the exemplified or envisaged glucose-binding agents (monomeric lectin domains, DNA aptamers, or peptide aptomers) restricted their placement to the N-terminal segment of the B chain as defined above. In the absence of exogenous glucose or other exogenous saccharide, intramolecular interactions between the A1-linked affinity ligand and B1-linked glucose-binding agent was envisaged to “close” the structure of the hormone and thereby impair its activity. Only modest glucose-responsive properties of this class of molecular designs were reported. In this class of analogues the B1-linked agents are typically as large or larger than insulin itself.

The suboptimal properties of insulin analogues modified at or near residue A1 by an affinity ligand and simultaneously modified at or near residue B1 by a large glucose-binding agent (i.e., of size similar or greater than that of an insulin A or B chain) are likely to be intrinsic to this class of molecular designs. Overlooked in the above class of insulin analogues are the potential advantages of an alternative type of glucose-regulated switch engineered exclusively within the B chain without modification of its amino-terminus and without the need for large domains unrelated in structure or composition to insulin. The insulin analogues of the present invention thus conform to one of four design schemes sharing the properties that (a) in the absence of glucose the modified insulin exhibits marked impairment in binding to the insulin receptor whereas (b) in the presence of a high concentration of glucose breakage of a covalent bond to a diol-modified B chain either leads to an active hormone conformation or liberates an active hormone analogue. Modifications of the insulin molecule are in each case smaller than the native A or B chains.

Surprisingly, we have found that this fundamentally different class of molecule designs may optimally provide a glucose-dependent conformational switch between inactive and active states of the insulin molecule without the above disadvantages. Whereas previous strategies to achieve such a switch employed a single diol-containing side chain in the B chain, the present invention focuses on main-chain atoms as an attachment point for hydroxyl groups comprising one or more diol moieties (FIG. 3C). These novel B-chain derivatives offer the mechanistic advantage of an immediate connection to the three-dimensional structure of wild-type insulin and inactive single-chain insulins: the main-chain hydroxyl groups more closely recapitulate a native salt bridge between the C terminus of the B chain and N terminus of the A chain (as in a subset of crystal structures of wild-type insulin) or a peptide bind between the C terminus of the B chain and N terminus of the A chain. In the present embodiments the salt bridge or peptide bond would be replaced by a non-covalent interaction or covalent bond between the main-chain-directed diol moiety (or moieties) and a glucose-binding element linked to the A chain at or near its N terminus. Two or more diol moieties in the B chain (such as a main-chain-directed diol and modification of a preceding side chain) may act together to enhance formation of a tether between the A- and B chains that impairs binding to the insulin receptor. Two or more diol moieties may also introduce cooperativity in the reaction of free glucose to break the tether through competitive binding to the glucose-binding element. An example of a side-chain diol is provided by L- or D-Dopa (FIG. 4A), an analogue of Phenylalanine or Tyrosine (FIG. 4B).

The insulin analogues of the present invention can be used in therapeutic pharmaceutical formulations. We envisage that such an insulin analogue formulation would be compatible with multiple devices (such as insulin vials, insulin pens, and insulin pumps) and could be integrated with modifications to the insulin molecule known in the art to confer rapid-, intermediate-, or prolonged insulin action. In addition, the present glucose-regulated conformational switch in the insulin molecule, engineered between the C-terminus of the B chain and N-terminus of the A chain, could be combined with other glucose-responsive technologies (such as closed-loop systems or glucose-responsive polymers) to optimize their integrated properties. We thus envisage that the products of the present invention will benefit patients with either Type 1 or Type 2 diabetes mellitus both in Western societies and in the developing world.

### SUMMARY

In accordance with one embodiment insulin analogues are provided that are inactive or exhibit reduced, prolonged activity under hypoglycemic conditions but are activated at high glucose concentrations for binding with the insulin receptor with high affinity. This transition exploits the use of diol moieties added to the carboxy terminus of the B chain such that at least one hydroxyl group is attached to the C-terminal main-chain atom of the B chain. The insulin analogues of the present disclosure contain two elements. The first element is a diol-containing side chain in the B chain; the second is a glucose-binding element attached at or near the N terminus of the A chain. This overall scheme is shown in FIG. 3C.

One aspect of the present disclosure is directed to glucose-responsive insulins containing novel B-chain analogues comprising one or more diols, and a glucose-binding element of arbitrary chemical composition at or near the N-terminus of the A chain. As disclosed herein design strategies and chemical approaches are described for synthesis of B-chain analogues that contain diol moieties positioned at a combination of main-chain and side-chain positions at or near the C terminus of the B chain. Whereas past design schemes have focused exclusively on diol modification of side chains, one aspect of the novel compositions disclosed herein relates to the use of main-chain-attached diols, either alone or in combination with conventional side-chain modifications. In one embodiment the B chain of the present invention has the standard 30 residues. In an alternative embodiment the insulin B chain differs from the native insulin B chain by the deletion of residues B30, B29-B30, B28-B30 or B27-B30; or an extension of additional residue B31 or additional residues B31-B32.

In accordance with one embodiment an insulin B chain is provided comprising residues B1-B26 of native insulin and a modified amino acid covalently linked to the C-terminus of the B chain via an amide bond, wherein the modified amino acid comprises a diol. Exemplary diol bearing amino acids/amino acid derivatives suitable for use in accordance with the present disclosure are a shown in FIGS. 7, 8 and 9. In one embodiment the variant B chain comprises a diol group at the C terminus of the B chain such that the polypeptide chain ends with a hydroxyl group rather than with a carboxylate group. This variant B chain can be used in conjunction with an insulin A chain that has been modified by the attachment of a glucose-binding element at the N-terminus of the A chain. In one embodiment an insulin analogue is provided comprising an A chain modified by a glucose-binding element at or near its N terminus and a variant B chain comprising a diol group at the C terminus of the B chain such that the polypeptide chain ends with a hydroxyl group rather than with a carboxylate group. The insulin A and B chains can be further modified to incorporate further advantageous substitutions that are known to the skilled practitioner to improve solubility or stability of the insulin analog. In accordance with one embodiment an insulin analogue is provided wherein the diol group at the C terminus of the B chain is an aliphatic (1, 2) diol. In another embodiment an insulin analogue is provided wherein the diol group at the C terminus of the B chain is an aliphatic (1, 3) diol.

In accordance with one embodiment an insulin B chain is provided comprising residues B1-B26 of native insulin and a modified amino acid covalently linked to the C-terminus of the B chain via an amide bond, wherein the B chain is further modified to comprise an additional modified amino acid at a position 1, 2, 3, or 4 residues N-terminal to the C-terminal amino acid, wherein said additional modified amino acid is an L or D amino acid comprising a side-chain diol. In one embodiment the additional modified amino acid is a thiol-containing L or D amino acid. In one embodiment the additional modified amino acid is an L Dopa at position B26 or an L or D Dopa located at 1-3 residues N-terminal to the C-terminal amino acid.

In accordance with one embodiment an insulin B chain is provided wherein the B chain is a truncated B chain lacking residue B30, residues B29-B30, residues B28-B30, residues B27-B30 or residues B26-B30, and further provided with a diol group located at the C terminus of the truncated B chain. In another embodiment an insulin B chain is provided wherein said B chain is extended by one or two amino acids with a diol group located at the C terminus of the extended B chain. In one embodiment the B chain is a polypeptide selected from the group consisting of

(SEQ ID NO: 1)
FVKQHLCGSHLVEALYLVCGERGFFYTEKX₃₀,
(SEQ ID NO: 2)
FVNQHLCGSHLVEALYLVCGERGFFYTDKX₃₀,
(SEQ ID NO: 3)
FVNQHLCGSHLVEALYLVCGERGFFYTKPX₃₀,
(SEQ ID NO: 4)
FVNQHLCGSHLVEALYLVCGERGFFYTPKX₃₀
(SEQ ID NO: 5)
FVNQHLCGSHLVEALYLVCGERGFFYTKX₃₀,
(SEQ ID NO: 6)
FVNQHLCGSHLVEALYLVCGERGFFYTPX₂₉X₃₀,
(SEQ ID NO: 7)
FVNQHLCGSHLVEALYLVCGERGFFYTPX₃₀
(SEQ ID NO: 8)
FVNQHLCGSHLVEALYLVCGERGFFYTX₂₉X₃₀
(SEQ ID NO: 9)
FVNQHLCGSHLVEALYLVCGERGFFYTX₃₀
and
(SEQ ID NO: 10)
FVNQHLCGSHLVEALYLVCGERGFFYX₃₀,

wherein
X₂₉ is ornithine; and
X₃₀ is a diol bearing amino acid derivative, optionally threoninol. In one embodiment of the present disclosure, the B chain is a polypeptide selected from the group consisting of

(SEQ ID NO: 37)
FVNQHLCGSHLVEALYLVCGERGFF[Sar][APD],
(SEQ ID NO: 38)
FVNQHLCGSHLVEALYLVCGERGFF[dA][APD],
and
(SEQ ID NO: 39)
FVNQHLCGSHLVEALYLVCGERGFFYG[APD],

wherein APD is 3-amino-1,2-propandiol.

In one embodiment of the present disclosure, the B chain is a polypeptide selected from the group consisting of

(SEQ ID NO: 11)
FVNQHLCGSHLVEALYLVCGERGFFYTDKX₃₁X₃₀,
(SEQ ID NO: 12)
FVNQHLCGSHLVEALYLVCGERGFFYTKPX₃₁X₃₀,
(SEQ ID NO: 13)
FVNQHLCGSHLVEALYLVCGERGFFYTPKX₃₁X₃₀,
(SEQ ID NO: 14)
FVNQHLCGSHLVEALYLVCGERGFFYTDKX₃₁X₃₂X₃₀,
(SEQ ID NO: 15)
FVNQHLCGSHLVEALYLVCGERGFFYTKPX₃₁X₃₂X₃₀
and
(SEQ ID NO: 16)
FVNQHLCGSHLVEALYLVCGERGFFYTPKX₃₁X₃₂X₃₀,

wherein
X₃₁ and X₃₂ are independently any amino acid; and
X₃₀ is a diol bearing amino acid derivative, optionally threoninol.

In one embodiment an insulin analog is provided comprising a B chain and an A chain, wherein the B chain comprises any of the diol bearing B chain analogs disclosed herein and the A chain is a polypeptide selected from the group consisting of

(SEQ ID NO: 17)
R-GIVEQCCTSICSLYQLENYCN;
and
(SEQ ID NO: 18)
R-GIVEQCCHSICSLYQLENYCN,

wherein
R is

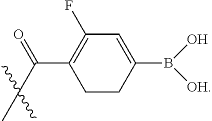 [SMILES: *C(=O)C1=C(F)C=C(B(O)O)CC1]

The present disclosure is also directed to a method of preparing the novel B chain analogs disclosed herein. A general molecular scheme is disclosed wherein a modified amino acid or non-acidic analogue (such as Threoninol instead of Threonine) is placed at or near the disordered carboxy-terminus of the B chain, such as at one of residues B27, B28, B29, B30 or within an extended B chain (i.e., residues B31, B32 or B33).

### BRIEF DESCRIPTION OF THE DRAWINGS

FIG. 1A is a schematic representation of the sequence of human proinsulin (SEQ ID NO: 41) including the A- and B-chains and the connecting region shown with flanking dibasic cleavage sites (filled circles) and C-peptide (open circles).

FIG. 1B is a structural model of proinsulin, consisting of an insulin-like moiety and a disordered connecting peptide (dashed line).

FIG. 1C is a schematic representation of the sequence of human insulin including the A-chain (SEQ ID NO: 43) and the B-chain (SEQ ID NO: 42) and indicating the position of residues B27 and B30 in the B-chain.

FIG. 2 is cylinder model of insulin in which the side chains of Tyr^{B16}, Phe^{B25} and Tyr^{B26} are shown. The A- and B-chain ribbons are shown in light gray and dark gray, respectively.

FIG. 3A provides a ribbon/cylinder diagram highlighting a potential salt bridge between the C-terminal carboxylate of the B chain (its negative charge is depicted as a—within circle) and alpha-amino group of the A chain (its positive charge is depicted as a + within circle) as observed in a subset of wild-type insulin crystallographic protomers.

FIG. 3B provides a ribbon/cylinder diagram highlighting a peptide bond (box) between the C-terminal carboxylate of the B chain and alpha-amino group of the A chain as observed in inactive single-chain insulin analogues.

FIG. 3C provides a generic scheme in which a diol-modified B chain containing a main-chain hydroxyl group (boxed) in combination with a neighboring hydroxyl group (which may be on a side chain or attached via one or more intervening atoms to the main-chain nitrogen) binds to a glucose-binding element at or near the N terminus of the A chain.

FIG. 4 provides line drawings of L-Dopa, phenylboronic acid, phenylalanine and tyrosine as free amino acids.

FIG. 5 illustrates the use of a C-terminal Threoninol to provide a combination of a main-chain-directed hydroxyl group and companion side-chain hydroxyl group to provide an aliphatic (1, 3) diol moiety on the B chain (SEQ ID NO: 46) with an accompanying A chain (SEQ ID NO: 47).

FIG. 6 illustrates a semi-synthetic scheme to prepare insulin analogues containing full-length (SEQ ID NO: 44) or truncated B chains (SEQ ID NO: 48) modified by a C-terminal Threoninol as depicted in FIG. 5. Capital O indicates ornithine (Orn) as a basic analogue of Lysine not susceptible to cleavage by trypsin. (A chain: (SEQ ID NO: 45)).

FIG. 7 depicts stereo-isomers of Threoninol that alter the spatial orientation of the hydroxyl groups relative to the main chain of the protein.

FIG. 8 provides the structure of a C-terminal Threoninol (a (1, 3) aliphatic diol), a (1, 2) diol APD and the structure of a complex between a boronic acid and a C-terminal diol (right at bottom).

FIG. 9 presents of series of alternative C-terminal main-chain-directed diols, triol or polyol suitable for use in accordance with the present invention.

FIG. 10 provides a synthetic scheme for a peptide containing homo-Tyr at the penultimate position and C-terminal APD diol. The methylene insertion in the penultimate side chain changes the position of the aromatic hydroxyl substituent.

FIG. 11 depicts a “wall” of a (1, 2) aliphatic diol element based on a shared framework derived from des-pentapeptide insulin (DPI).

FIG. 12 depicts potential glucose sensors containing two phenyl-boronic acids.

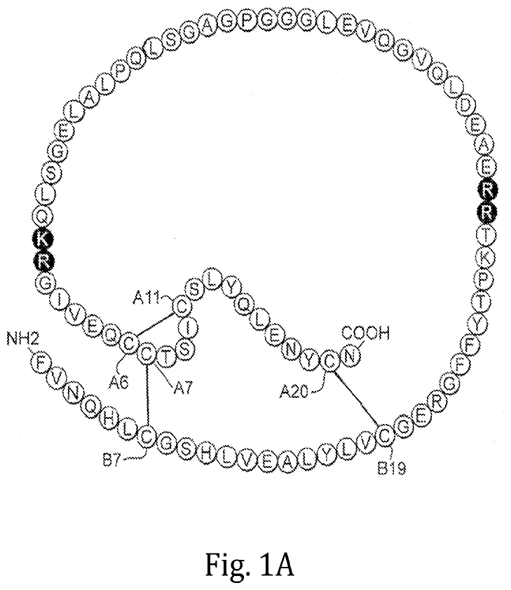

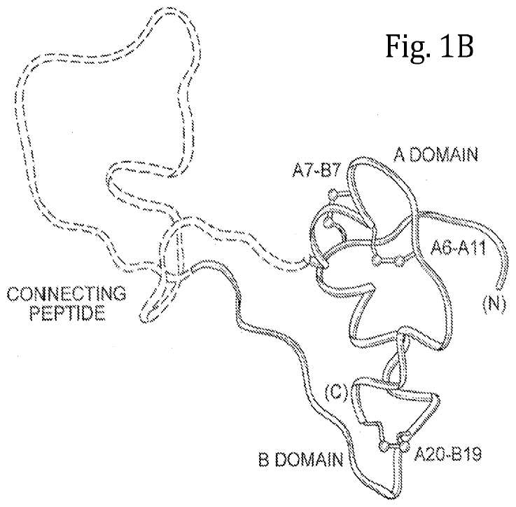

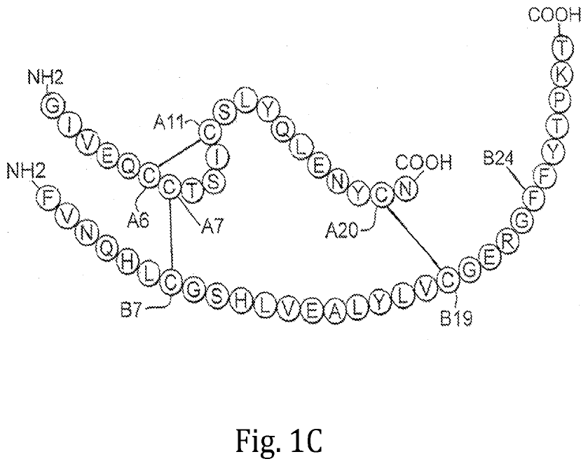

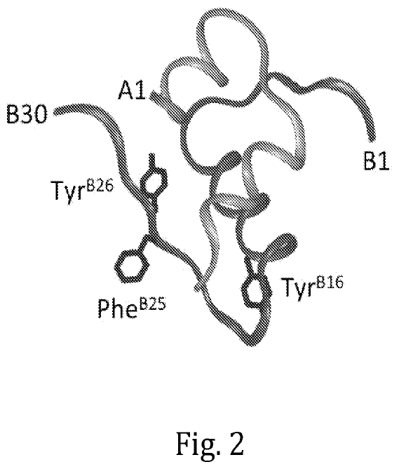

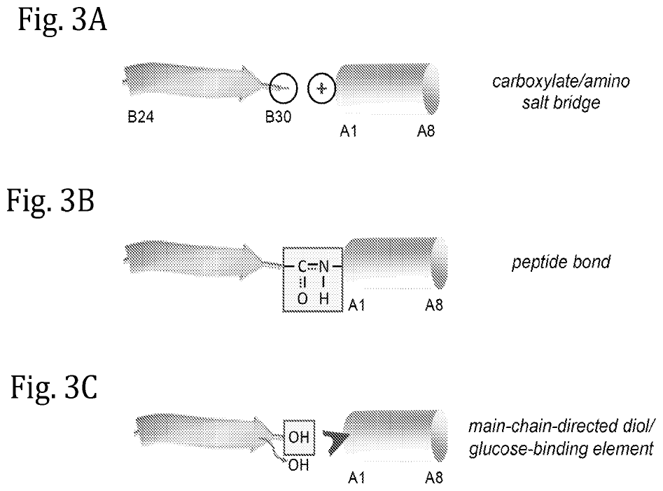

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “patient” without further designation is intended to encompass any warm blooded vertebrate domesticated animal (including for example, but not limited to livestock, horses, cats, dogs and other pets) and humans receiving a therapeutic treatment with or without physician oversight.

The term “inhibit” defines a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, the term “threoninol” absent any further elaboration encompasses L-allo-threoninol, D-threoninol and D-allo-threoninol.

As used herein the term “main-chain” defines the backbone portion of a polypeptide, and distinguishes the atoms comprising the backbone from those that comprise the amino acid side chains that project from the main-chain.

As used herein, the term “pharmaceutically acceptable salt” refers to those salts with counter ions which may be used in pharmaceuticals. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. Such salts include:
(1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D)- or (L)-malic acid, maleic acid, methane sulfonic acid, ethanesulfonic acid, p-toluenesulfonic acid, salicylic acid, tartaric acid, citric acid, succinic acid or malonic acid and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Acceptable salts are well known to those skilled in the art, and any such acceptable salt may be contemplated in connection with the embodiments described herein. Examples of acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable acceptable salts are found in Remington's Pharmaceutical Sciences, 17th Edition, Mack Publishing Company, Easton, Pa., 1985.

### Representative Embodiments

The present disclosure relates to polypeptide hormone analogues that contain a glucose-regulated molecular structure or glucose-detachable molecular moiety, designed respectively either (a) to confer glucose-responsive binding to cognate cellular receptors and/or (b) to enable glucose-mediated liberation of an active insulin analogue. More particularly, the present disclosure is directed to insulin analog that are responsive to blood glucose levels and their use in the treatment of patients and non-human mammals with Type 1 or Type 2 diabetes mellitus by subcutaneous, intraperitoneal or intravenous injection of the insulin analogs disclosed herein.

The insulin analogues of the present invention may also exhibit other enhanced pharmaceutical properties, such as increased thermodynamic stability, augmented resistance to thermal fibrillation above room temperature, decreased mitogenicity, and/or altered pharmacokinetic and pharmacodynamic properties. More particularly, this disclosure relates to insulin analogues that may confer either rapid action (relative to wild-type insulin in its regular soluble formulation), intermediate action (comparable to NPH insulin formulations known in the art) or protracted action (comparable to basal insulins known in the art as exemplified by insulin detemir and insulin glargine) such that the affinity of the said analogues for the insulin receptor is higher when dissolved in a solution containing glucose at a concentration above the physiological range (>140 mg/dl; hyperglycemia) than when dissolved in a solution containing glucose at a concentration below the physiological range (<80 mg/dl; hypoglycemia).

In accordance with one embodiment an insulin analogue is provided comprising an A chain modified by a glucose-binding element at or near its N terminus and a variant B chain comprising a diol group at the C terminus of the B chain such that the polypeptide chain ends with a hydroxyl group rather than with a carboxylate group. Reduced or absent activity is associated with formation of a covalent bond between the unique diol moiety in the B chain and a second molecular entity located at the N-terminus of the A chain and that contains a glucose-binding element. Displacement of the B chain diol from the A-chain-linked glucose-binding element by glucose would lead to detachment of the tethered molecular entity, which in turn enables high-affinity receptor binding. In the absence of glucose the C-terminus diols remain bound to the A-chain-linked glucose-binding element and the insulin analog remains inactive.

In accordance with one embodiment the modified B chain may contain a broad molecular diversity of diol-containing moieties (or adducts containing an α-hydroxycarboxylate group as an alternative binding motif that might bind to a glucose-binding element), whether a saccharide or a non-saccharide reagent. Possibilities include an N-linked or O-linked saccharide or any organic moiety of similar molecular mass that contains a diol function that mimics the diol function of a monosaccharide and hence confers reversible PBA-binding activity (or adducts containing an α-hydroxycarboxylate group as an alternative PBA-binding function; PBA in the present invention may equivalently be substituted by other boron-containing diol-binding elements as known in the art to bind glucose). Such non-saccharide diol-containing organic compounds span a broad range of chemical classes, including acids, alcohols, thiol reagents containing aromatic and non-aromatic scaffolds; adducts containing an α-hydroxycarboxylate group may provide an alternative function able to bind PBA or other boron-containing diol-binding elements able to bind glucose. Convenient modes of attachment to the B chain also span a broad range of linkages in addition to the above N-linked and O-linked saccharide derivatives described above; these additional modes of attachment include (i) the side-chain amino function of Lysine, ornithine, diamino-butyric acid, diaminopropionic acid (with main-chain chirality L or D) and (ii) the side-chain thiol function of Cysteine or homocysteine (with main-chain chirality L or D). A preferred embodiment at sites of native aromatic acids (positions B16, B25 and B26) is provided by L-Dopa.

The molecular purpose of the diol-modified B chain is to form an intramolecular bond or bonds with the A-chain-attached glucose-binding element such that the conformation of insulin is “closed” and so impaired in binding to the insulin receptor. Use of a main-chain-directed diol recapitulates the inactive structure of a single-chain insulin analogue. We envision that at high glucose concentrations, the diol-glucose-binding element bond or bonds will be broken due to competitive binding of the glucose to the glucose-binding element. Preferred embodiments contain two or more diol groups in an effort to introduce cooperativity. The main-chain element can be via substitution of the C-terminal carboxylate by a hydroxyl group together with an appropriately positioned side-chain hydroxyl group and/or via a moiety attached to the main-chain nitrogen atom.

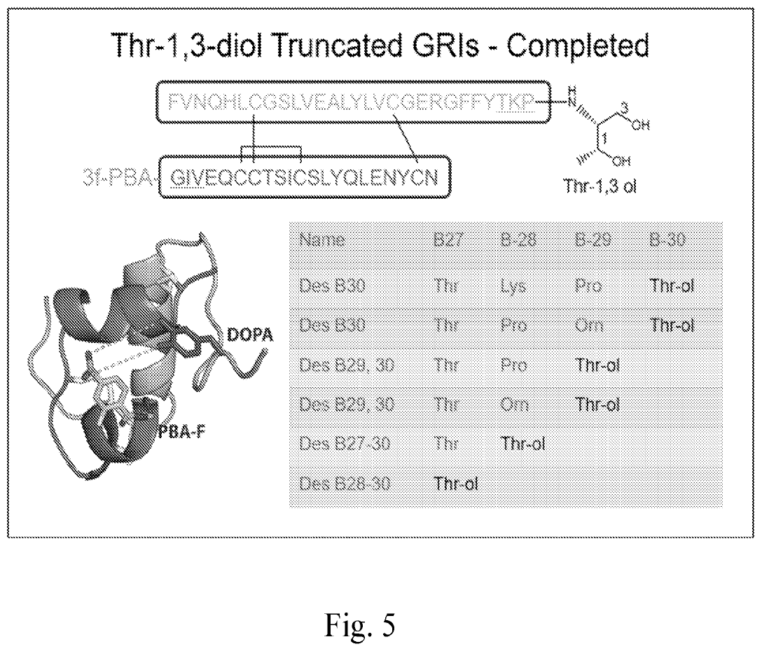

An aspect of the present invention provides a new approach that couples either an aliphatic (1,3)-diol or an aliphatic (1,2) diol as B-Chain C-terminal carboxamides octapeptides to produce new chemical entities representing either full length insulin analogues or truncated insulin analogues with diol groups at the B-chain C-terminus. A logical set of B-chain analogues containing C-terminal L-Threoninol residues (as a representative (1, 3) aliphatic diol) is shown in FIG. 5.

### Synthetic Approach.

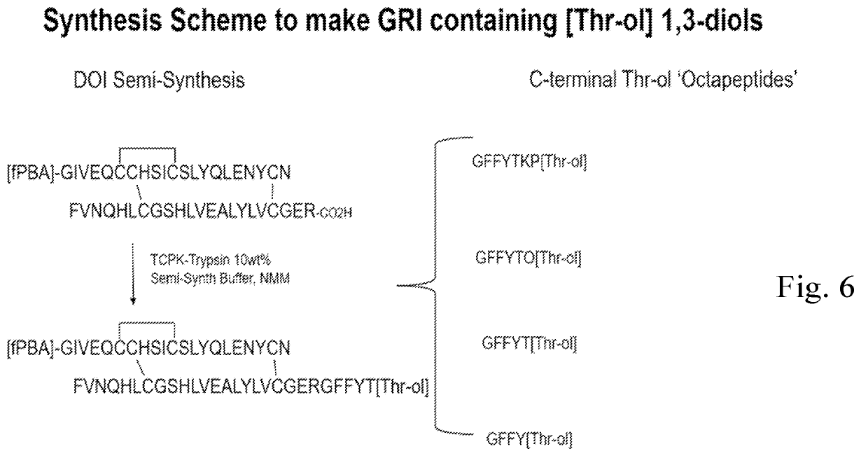

We prepared all the GRI compounds by the trypsin mediated semi-synthesis approach that brings together a separately purified des octapeptide insulin [DOI] precursor that has a model diol-binding element (namely, meta-fluoro-4-carboxylphenylboronic acid [meta-fPBA or m-fPBA]) at the A-chain N-terminal of a previously folded insulin (i.e., His^{AB}-insulin skeleton). This DOI meta-fPBA-A1 His^{AB} DOI served as the C-terminal donor in in the trypsin mediate synthesis while the diol containing octapeptide surrogates serve as the amino donor in the reaction. This scheme is shown in FIG. 6 in relation to (1, 2) aliphatic diols. We emphasize that the scope of the present invention is not restricted to meta-fPBA, employed herein only as an illustrative glucose-binding element. His^{A8} was incorporated to enhance affinity of the analogue for the insulin receptor, otherwise mildly impaired by the meta-fPBA-A1 adduct.

(1, 3) diol Proposed Design Strategies:

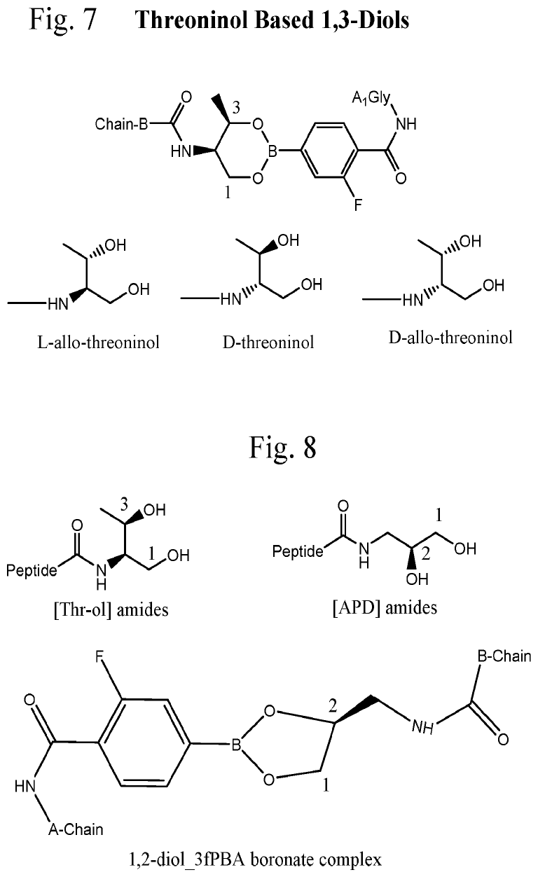

We prepared seven Threonine-based (1, 3) diol octapeptide surrogates by incorporating L-Threoninol [Thr-ol] [Cas, 3228-51-1] at the B-chain C-terminal. Our initial focus sources commercially available Threonine derived aliphatic 1,3-diol [Thr-ol] as the Fmoc-Thr(OtBu)-CH2O-ClTrt-resin which was used in peptide synthesis to produce 8mer peptides, i.e., GYYFTTKP[Thr-ol] (SEQ ID NO: 40) and systematic truncated analogues down to 4mers GFFY[Thr-ol] (SEQ ID NO: 22) to place the diol at B27, B28, B29, B30 positions. See below for the completed synthesis of the Thr-ol (1, 3)-diol truncated GRI series (See below). Note that L-Threoninol has two chiral centers, so it is possible to use L-allo-Threoninol (Cas 108102-48-3), D-Threoninol (cas #44520-55-0), or D-allo-Threoninol to evaluate these positions for activity. These stereo-isomers are shown in FIG. 7. The set of synthetic peptides employed in the semi-synthetic reactions is given in Table 1.

TABLE 1
1,3-Diol: Threoninol Octapeptide Surrogates

| Notebook # | Description | Sequence | Diol Position |
|---|---|---|---|
| MJ01-62-02 | [Thr-ol]B30 KP octapeptide | GFFYTKP[Thr-ol] | B30 |
| | | SEQ ID NO: 19 | |
| MJ01-62-04 | [Thr-ol]B29 heptapeptide | GFFYTK[Thr-ol] | B29 |
| | | SEQ ID NO: 20 | |
| MJ01-63-02 | [Thr-ol]B28 hexapeptide | GFFYT[Thr-ol] | B28 |
| | | SEQ ID NO: 21 | |
| MJ01-63-04 | [Thr-ol]B27 hexapeptide | GFFY[Thr-ol] | B27 |
| | | SEQ ID NO: 22 | |
| MJ01-68-01 | OrnB28, [Thr-ol]B29 | GFFYTO[Thr-ol] | B29 |
| | | SEQ ID NO: 23 | |
| MJ01-68-03 | [Thr-ol]B29 octapeptide | GFFYTP[Thr-ol] | B29 |
| | | SEQ ID NO: 24 | |
| MJ01-69-01 | [Thr-ol]B30 POT octapeptide | GFFYTPO[Thr-ol] | B30 |
| | | SEQ ID NO: 25 | |

In another approach we incorporated (S)-3-amino-1,2-propandiol [(s)-APD] as an aliphatic (1, 2) vicinal diol into the B-chain C-terminal (FIG. 8, top right). The (s)-APD group was linked as the C-terminal amide and represents B-chain truncated modified insulin analogues (FIG. 8). (We note that (R)-3-amino-1,2-propandiol could also be incorporated.) The (S)-3-amino-1,2-propandiol [(s)-APD] derived-terminal peptide amides carrying the aliphatic vicinal 1,2-diols were prepared from N-terminal N-Boc protected peptide sub-assembly peptide using standard solution phase amide bond carbodiimide (EDCI, 6-Cl-HOBT or DIC) coupling reactions and (S)-(+)-(2,2-dimethyl-[1,3]-dioxolan-4-yl)-methylamine as the amine component. A series of such modified synthetic peptides is given in Table 2. Candidate insulin analogues are given in Table 3 (for brevity, His^{AB} is denoted “HA8” in Table 3, and likewise Gly^{B27} is denoted “GB27” and Orn^{B28} is denoted 0B28); as above, the phenyl-boronic acid moiety was employed only as a model glucose-binding element known in the art (bottom panel of FIG. 8) and is not meant to restrict the scope of the present invention.

TABLE 2
1,2-diols: (S)-3-Amino-1,2-Propandiol (s)-APD C-terminal Amides

| Notebook # | Description | Sequence | MW Th. | Observed MS lons [m/z] | Rt [min] | Purity |
|---|---|---|---|---|---|---|
| MJ02-04-03 | [APD]B27 | GFFY[APD] | 605.60 | 606.2, 1211.3 (dimer) | 21.84 | >95% |
| | tetrapeptide | SEQ ID NO: 26 | | | | |
| MJ02-04-01 | sub-assembly | Boc-GFFY(tBu)-OH | 688.70 | Not characterized | | |
| | | SEQ ID NO: 27 | | | | |
| MJ02-12-01 | [APD]B28 | GFFYG[APD] | 662.74 | 663.2, 1325.4 (dimer) | 20.31 | >95% |
| | pentapeptide | SEQ ID NO: 28 | | | | |
| MJ02-09-02 | sub-assembly | Boc-GFFY(tBu)G-OH | 745.77 | 746.2, 1491.4 (dimer) | 26.46 | >95% |
| | | SEQ ID NO: 29 | | | | |
| MJ02-10-04 | [APD]B28 | GFFYT[APD] | 706.80 | Not characterized | | >95% |
| | pentaptide | SEQ ID NO: 30 | | | | |
| MJ01-93-03 | sub-assembly | Boc-GFFY(tBu)T(tBu)-OH | 845.90 | 846.2, 1692.2 (dimer) | 29.5 | >95% |
| | | SEQ ID NO: 31 | | | | |
| MJ02-18-01 | [APD]B29 | GFFYTG[APD] | 763.85 | 764.3, 1527.4 (dimer) | 20.8 | >95% |
| | hexapeptide | SEQ ID NO: 32 | | | | |
| MJ02-13-02 | sub-assembly | Boc-GFFY(tBu)T(tBu)G-OH | 902.87 | Not characterized | | |
| | | SEQ ID NO: 33 | | | | |
| MJ02-18-03 | [APD]B27 | GFF[Sar] [APD] | 513.59 | 514.2, 1027.33 (dimer) | 18.61 | >95% |
| | tetrapeptide | SEQ ID NO: 34 | | | | |
| MJ02-13-04 | sub-assembly | Boc-GFF[Sar]-OH | 540.25 | 541.1, 1081.2 (dimer | 35.18 | >95% |
| | | SEQ ID NO: 35 | | | | |
| MJ02-19-01 | [APD]B27 | GFF[D-Ala] [APD] | 513.59 | 514.2, 1027.3 | 20.14 | >95% |
| | tetrapeptide | SEQ ID NO: 36 | | | | |

HPLC method : Peptides were purified by preparative RP-HPLC on a CLIPEUS C8 (20 × 250 mm, 5 um, Higgins Analytical) column with 0.1% TFA/H2O (A) and 0.1% TFA/CH3CN (B) as elution buffers. Identity of the peptides was confirmed by LC-MS (Finnigan LCQ Advantage, Thermo) on a TARGA C8 (4.6 x 250 mm, 5 um, Higgins Analytical) with 0.1% TFA/H2O (A) and 0.1% TFA/CH3CN as eluents using a gradient method of 5-45%B over 45 min.

TABLE 3
1,3-[Thr-ol] and 1,2-[APD] Containing GRIs

| Notebook # | Description | Sequence |
|---|---|---|
| MJ01-64-01 | HA8, [Thr-ol]B30, | FVNQHLCGSHLVEALYLVCGERGFFYTKP[Thr-ol] (SEQ ID NO: 3)---[fPBA]- |
| | KP-GRI | GIVEQCCHSICSLYQLENYCN (SEQ ID NO: 18) |
| MJ01-65-01 | HA8, [Thr-ol]B29, | FVNQHLCGSHLVEALYLVCGERGFFYTK[Thr-ol] (SEQ ID NO: 5)---[fPBA]- |
| | KB29-GRI | GIVEQCCHSICSLYQLENYCN(SEQ ID NO: 18) |
| MJ01-70-01 | HA8, [Thr-ol]B28-GRI | FVNQHLCGSHLVEALYLVCGERGFFYT[Thr-ol] (SEQ ID NO: 9)---[fPBA]- |
| | | GIVEQCCHSICSLYQLENYCN(SEQ ID NO: 18) |
| MJ01-67-01 | HA8, [Thr-ol]B27-GRI | FVNQHLCGSHLVEALYLVCGERGFFY[Thr-ol] (SEQ ID NO: 10)---[fPBA]- |
| | | GIVEQCCHSICSLYQLENYCN(SEQ ID NO: 18) |
| MJ01-68-02 | HA8, OB28, [Thr-ol] | FVNQHLCGSHLVEALYLVCGERGFFYTO[Thr-ol] (SEQ ID NO: 8)---[fPBA]- |
| | B29-GRI | GIVEQCCHSICSLYQLENYCN (SEQ ID NO: 18) |
| MJ01-68-04 | HA8, [Thr-ol]B29-GRI | FVNQHLCGSHLVEALYLVCGERGFFYTP[Thr-ol] (SEQ ID NO: 7)---[fPBA]- |
| | | GIVEQCCHSICSLYQLENYCN(SEQ ID NO: 18) |
| MJ01-69-02 | H8A, OB29,[Thr-ol] | FVNQHLCGSHLVEALYLVCGERGFFYTPO[Thr-ol] (SEQ ID NO: 6)---[fPBA]- |
| | B30-GRI | GIVEQCCHSICSLYQLENYCN(SEQ ID NO: 18) |
| MJ02-28-01 | HA8, SarB26, ApdB27-GRI | FVNQHLCGSHLVEALYLVCGERGFF[Sar][APD] (SEQ ID NO: 37)--- |
| | | [fPBA]GIVEQCCHSICSLYQLENYCN(SEQ ID NO: 18) |
| MJ02-29-01 | HA8, dAB26, ApdB27-GRI | FVNQHLCGSHLVEALYLVCGERGFF[dA][APD] (SEQ ID NO: 38)--- |
| | | [fPBA]GIVEQCCHSICSLYQLENYCN(SEQ ID NO: 18) |
| MJ02-30-01 | HA8, GB27, ApdB28-GRI | FVNQHLCGSHLVEALYLVCGERGFFYG[APD] (SEQ ID NO: 39)--- |
| | | [[fPBA]GIVEQCCHSICSLYQLENYCN(SEQ ID NO: 18) |

Yet other alternative C-terminal poly-ol amides such as [Ser-ol], [Tris], [bis(hyroxyethyl)aminoethyl], [1,2,3-propane-triol], or [furanosyl-triol] amides can also be incorporated at the B-Chain C-terminus to produce full-length or truncated insulin analogues. These alternatives are depicted in FIG. 9.

### Placement of Diol Functionalities Along the Amide Backbone.

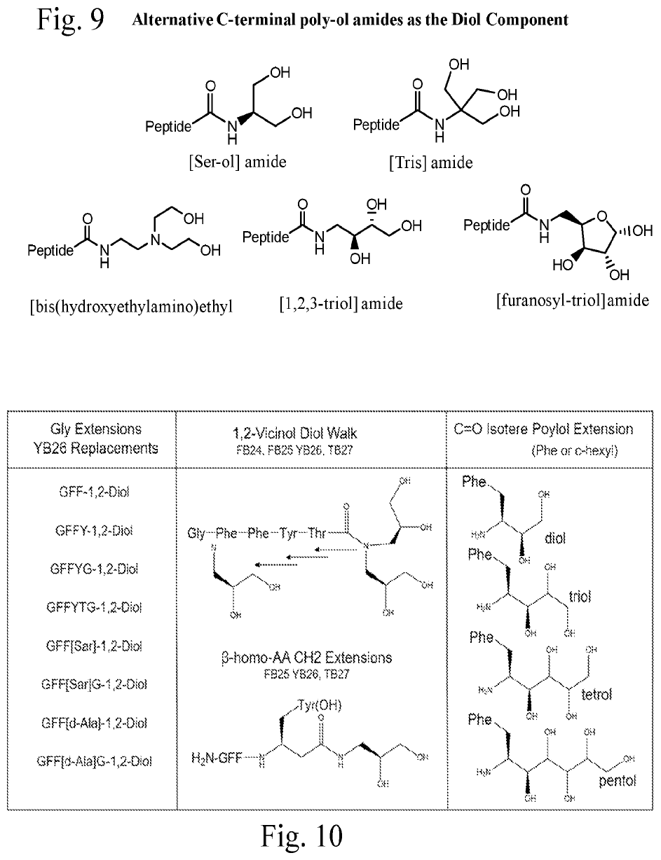

The main-chain amide nitrogen atom may be modified with the APD where the 3-amino group of 3-aminopropane-1,2-diol [APD] functions also as the amide nitrogen. Our synthetic strategy thus allows for APD placement at any amide position except for at a proline residue. Thus, in FIG. 10 (column 2, top panel) are illustrated a ‘walk’ of the (1, 2) vicinal diol to positions FB24, FB25, YB26, GB26 D-Ala^{B26}, but can also be extended to positions B27, thru B32 along with the C-terminal modified as 1,2 diol [APD] (preferred) or 1,3 [Thr-ol]. Placement of multiple amide-backbone diols is possible and may provide greater opportunity for forming boronate esters thus stabilizing a closed insulin state that opens upon exposure to glucose.

Other alternative backbone poly-ol amides are envisioned like [Ser-ol],[bis(hyroxyethyl)-aminoethyl], [1,2,3-propane-triol], or [furanosyl-triol] amides (shown above) which can be produced reductive alkylation from the corresponding aldehydes.

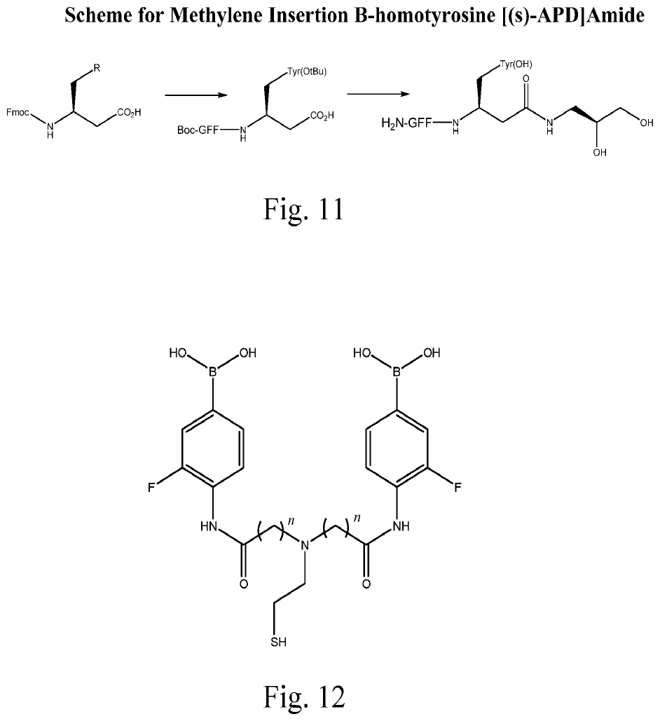

Also in FIG. 10 (column 2 in bottom panel) are illustrated beta-homo-amino acids by using β-homophenylalanine (FB25), β-homotyrosine (YB26), β-homothreonine (TB27), β-homoproline (PB28), or β-homolysine (KB29) provide opportunity to make a single methylene incorporation to tune positioning of both C-terminal diol and the amide backbone walking diol (see above). Using B-homo-amino acids provide increased flexibility and avoiding racemization when forming C-terminal [APD] and other poly-ol carboxamides. The synthetic scheme is illustrated in FIG. 11.

Further in FIG. 10 (column 3) is illustrated a strategy for B-chain C-terminal amino-polyol extension that also incorporate(s) configuration side-chain residue functionality while presenting diols, triols, tetrols, and pentols. Multiple poly-ol groups provide for greater opportunity to complex through multiple binding modes the PBA group(s). Chemistry methods to produce such dihydroxyethylene amino acid mimic is well established.

Alternative boron-based glucose-binding elements are known in the art, and they are not included within the scope of the present invention, except as unique combinations with the claimed B-chain analogues. Examples are provided in FIG. 12. Bisboronic acids have been shown to have glucose selectivity toward glucoses via cooperative effects. Such bisboronic acid molecular architectures have been used as glucose sensors and displayed higher glucose affinities compared to the monomeric forms (4-amino-3-fluorophenylboronic acid, and 3-carboxy-benzoboroxole). Sharma et al constructed 4-amino-3-fPBA sensors by sequential alkylation of a protected cysteamine precursor and subsequent carbodiimide (EDC)-promoted coupling of 4-amino-3-fluorophenylboronic acid. The analogues of the present invention may contain any glucose-binding element at or near the N terminus of the A chain and so are not restricted to such elements that may contain the element boron. The scope of the present disclosure includes a main-chain-directed diol in combination with a large chemical space of diol-containing compounds attached to a preceding side chain as listed in Table 4.

The analogues of the present invention may optionally contain an additional saccharide-binding element attached to residue B1 as a mechanism intended to provide glucose-sensitive binding of the insulin analogue to surface lectins in the subcutaneous depot. In addition, the analogues of the present invention may optionally contain substitutions known in the art to confer rapid action (such as Asp^{B28}, a substitution found in insulin aspart (the active component of Novolog®); [Lys^{B28}, Pro^{B29}], pairwise substitutions found in insulin lispro (the active component of Humalog®); Glu^{B29} or the combination [Lys^{B3}, Glu^{B29}] as the latter is found in insulin glulisine (the active component of Apridra®), or modifications at position B24 associated with accelerated disassembly of the insulin hexamer (e.g., substitution of Phe^{B24} by Cyclohexanylalanine or by a derivative of Phenylalanine containing a single halogen substitution within the aromatic ring). Alternatively, the analogues of the present invention may optionally contain modifications known in the art to confer protracted action, such as modification of the ε-amino group of Lys^{B29} by an acyl chain or acyl-glutamic acid adduct as respectively illustrated by insulin detemir (the active component of Levemir®) and insulin degludec (the active component of Tresiba®); or contain basic amino-acid substitutions or basic chain extensions designed to shift the isoelectric point (pI) to near neutrality as exemplified by the Arg^{B31}_Arg^{B32} extension of insulin glargine (the active component of Lantus®). Analogues of the present invention designed to exhibit such a shifted pI may also contain a substitution of Asn^{A21}, such as by Glycine, Alanine or Serine. Analogues of the present invention may optionally also contain non-beta-branched amino-acid substitutions of Thr^{A8} associated with increased affinity for the insulin receptor and/or increased thermodynamic stability as may be introduced to mitigate deleterious effects of the primary two above design elements (a phenylboronic acid derivative at or near the N-terminus of the A chain and one or more saccharide derivatives at or near the C-terminus of the B chain) on receptor-binding affinity and/or thermodynamic stability. Examples of such A8 substitutions known in the art are His^{A8}, Lys^{A8}, Arg^{A8}, and Glu^{A8}.

The insulin analogues of the present invention may exhibit an isoelectric point (pI) in the range 4.0-6.0 and thereby be amenable to pharmaceutical formulation in the pH range 6.8-7.8; alternatively, the analogues of the present invention may exhibit an isoelectric point in the range 6.8-7.8 and thereby be amenable to pharmaceutical formulation in the pH range 4.0-4.2. The latter conditions are known in the art to lead to isoelectric precipitation of such a pI-shifted insulin analogue in the subcutaneous depot as a mechanism of protracted action. An example of such a pI-shifted insulin analogue is provided by insulin glargine, in which a basic two-residue extension of the B chain (Arg^{B31}_Arg^{B32}) shifts the pI to near-neutrality and thus enables prolonged pharmacokinetic absorption from the subcutaneous depot. In general the pI of an insulin analogue may be modified through the addition of basic or acidic chain extensions, through the substitution of basic residues by neutral or acidic residues, and through the substitution of acidic residues by neutral or basic residues; in this context we define acidic residues as Aspartic Acid and Glutamic Acid, and we define basic residues as Arginine, Lysine, and under some circumstances, Histidine. We further define a “neutral” residue in relation to the net charge of the side chain at neutral pH.

It is an additional aspect of the present invention that absolute in vitro affinities of the insulin analogue for insulin receptor (isoforms IR-A and IR-B) are in the range 5-100% relative to wild-type human insulin and so unlikely to exhibit prolonged residence times in the hormone-receptor complex; such prolonged residence times are believed to be associated with enhanced risk of carcinogenesis in mammals or more rapid growth of cancer cell lines in culture. It is yet an additional aspect of the present invention that absolute in vitro affinities of the insulin analogue for the Type 1 insulin-like growth factor receptor (IGF-1R) are in the range 5-100% relative to wild-type human insulin and so unlikely either to exhibit prolonged residence times in the hormone/IGF-1R complex or to mediate IGF-1R-related mitogenesis in excess of that mediated by wild-type human insulin.

The insulin analogues of the present invention consist of two polypeptide chains that contain a novel modifications in the B chain such that the analogue, in the absence of glucose or other exogenous saccharide, contains covalent bonds between the side-chain diol in the B chain and a molecular entity containing PBA, a halogen-derivative of PBA, or any boron-containing diol-binding element able to bind glucose. The latter entity may be a C-terminal extension of the B chain or be a separate molecule prior to formation of the diol-PBA bonds.

Table 4 presents diol- or α-hydroxycarboxylate-containing precursors

TABLE 4

| | |
|---|---|
| 1,3-benzenedimethanol | 2,2,4,4-tetramethyl-1,3-cyclobutanediol |
| mannitol | butylboronic acid |
| fructose | isosorbide |
| sorbitol | N,N-dimethylsphingosine |
| Tris base | sphingosine (2-amino-4-octadecene-1,3- |
| Fmoc-3,4-dihydroxy-L-phenylalanine | diol) |
| 2-(acetoxymethyl)-4-iodobutyl acetate | tartaric acid |
| 1(1R,2S,3R,5R)-3-amino-5- | guaifenesin |
| (hydroxymethyl)-1,2-cyclopentanediol | 5β-Androstane-3α,17α-diol-11-one-17β- |
| hydrochloride | carboxylic acid 3-(β-D-glucuronide) |
| 2-(N-Fmoc-4-aminobutyl)-1,3-propanediol | (1S-cis)-3-bromo-3,5-cyclohexadiene-1,2- |
| 2-(4-aminobutyl)-1,3-propanediol | diol |
| 3-amino-1-,2-propandiol | dihydroxyphenylethylene glycol |
| 2-aminopropane-1,3-diol | |
| 3-mercaptopropane-1,2-diol | |
| 2-amino-4-pentane-1,3-diol | |
| N-acetyl-D-galactosamine | |
| N-acetylquinovosamine | |
| allopumiliotoxin 267A | |
| aminoshikimic acid | |
| atorvastatin | |
| β-D-galactopyranosylamine | |
| cafestol | |
| glafenine | |
| glyceraldehyde | |
| glyceric acid | |
| glycerol 3-phosphate | |
| glycerol monostearate | |
| hydrobromide | |
| 1,2,3,4-tetrahydro isoquinoline-6,7-diol | |
| D-sphingosine | |
| cyclohexane-1,2-diol | |
| cytosine glycol | |
| 4,5-dihydroxy-2,3-pentanedione | |
| dihydroxyphenylethylene glycol | |
| dithioerythritol | |
| dithiothreitol | |
| dropropizine | |
| dyphylline | |
| flavagline FL3 | |
| floctafenine | |
| (3S,4R)-4-methyl-5-hexene-1,3-diol | |
| (3S,4R)-4-Methyl-5-hexene-2,3-diol1,3 | |
| butanediol | |
| erithritol | |
| salicylhydroxamic acid | |
| catechol | |
| cis-1,2-cyclopentanediol | |
| cyclohexane-1,2-diol | |
| 1,2-dihydroxybenzene | |

Although we do not wish to be restricted by theory, we envisage that these two design elements form a covalent interaction in the absence of exogenous glucose such that the structure of the hormone is stabilized in a less active conformation.

In accordance with embodiment 1 an insulin analogue is provided comprising an A chain modified by a glucose-binding element at or near its N terminus and a variant B chain comprising a diol group at the C terminus of the B chain such that the polypeptide chain ends with a hydroxyl group rather than with a carboxylate group.

In accordance with embodiment 2 an insulin analogue of claim 1 is provided wherein the A chain contains a substitution at position A8 that enhances affinity of the insulin analogue for the insulin receptor, optionally wherein the substitution at position A8 is histidine.

In accordance with embodiment 3 an insulin analogue of embodiment 1 or 2 is provided wherein the A chain contains a substitution at position A8 or position A14 that enhances thermodynamic stability of the insulin analogue for the insulin receptor, optionally wherein the substitution at position A8 or A14 is independently selected from the group consisting of His^{A8}, Lys^{A}8, Arg^{A}8, and Glu^{A}8.

In accordance with embodiment 4 an insulin analogue of any one of embodiments 1-3 is provided wherein the A chain contains a substitution at position A21 that protects the insulin analogue from chemical degradation.

In accordance with embodiment 5 an insulin analogue of any one of embodiments 1-4 is provided wherein said diol group at the C terminus of the B chain is an aliphatic (1, 2) diol.

In accordance with embodiment 6 an insulin analogue of any one of embodiments 1-5 is provided wherein said diol group at the C terminus of the B chain is an aliphatic (1, 3) diol.

In accordance with embodiment 7 an insulin analogue of any one of embodiments 1-6 is provided further comprising a modified amino acid at a position 1, 2, 3, or 4 residues N-terminal to the C-terminal amino acid, wherein said modified amino acid is an L or D amino acid comprising a side-chain diol.

In accordance with embodiment 8 an insulin analogue of any one of embodiments 1-7 is provided wherein the modified amino acid is thiol-containing L or D amino acid.

In accordance with embodiment 9 an insulin analogue of any one of embodiments 1-8 is provided further comprising an L Dopa at position B26 or an L or D Dopa located at 1-3 residues N-terminal to the C-terminal amino acid.

In accordance with embodiment 10 an insulin analogue of any one of embodiments 1-9 is provided wherein said B chain is a truncated B chain lacking residue B30, residues B29-B30, residues B28-B30, residues B27-B30 or residues B26-B30, with a diol group located at the C terminus of the truncated B chain.

In accordance with embodiment 11 an insulin analogue of any one of embodiments 1-9 is provided wherein said B chain is extended by one or two amino acids with a diol group located at the C terminus of the extended B chain.

In accordance with embodiment 12 an insulin analogue of any one of embodiments 1-9 is provided wherein the B chain is a polypeptide selected from the group consisting of

(SEQ ID NO: 1)
FVKQHLCGSHLVEALYLVCGERGFFYTEKX₃₀,
(SEQ ID NO: 2)
FVNQHLCGSHLVEALYLVCGERGFFYTDKX₃₀,
(SEQ ID NO: 3)
FVNQHLCGSHLVEALYLVCGERGFFYTKPX₃₀,
(SEQ ID NO: 4)
FVNQHLCGSHLVEALYLVCGERGFFYTPKX₃₀
(SEQ ID NO: 5)
FVNQHLCGSHLVEALYLVCGERGFFYTKX₃₀,
(SEQ ID NO: 6)
FVNQHLCGSHLVEALYLVCGERGFFYTPX₂₉X₃₀,
(SEQ ID NO: 7)
FVNQHLCGSHLVEALYLVCGERGFFYTPX₃₀
(SEQ ID NO: 8)
FVNQHLCGSHLVEALYLVCGERGFFYTX₂₉X₃₀
(SEQ ID NO: 9)
FVNQHLCGSHLVEALYLVCGERGFFYTX₃₀
and
(SEQ ID NO: 10)
FVNQHLCGSHLVEALYLVCGERGFFYX₃₀,

wherein
X₂₉ is ornithine; and
X₃₀ is a diol bearing amino acid derivative, optionally threoninol. In one embodiment 13, an insulin analogue of any one of embodiments 1-9 is provided wherein the B chain is a polypeptide selected from the group consisting of

(SEQ ID NO: 37)
FVNQHLCGSHLVEALYLVCGERGFF[Sar][APD],
(SEQ ID NO: 38)
FVNQHLCGSHLVEALYLVCGERGFF[dA][APD],
and
(SEQ ID NO: 39)
FVNQHLCGSHLVEALYLVCGERGFFYG[APD],

In accordance with embodiment 14 an insulin analogue of any one of embodiments 1-9 is provided wherein the B chain is a polypeptide selected from the group consisting of

(SEQ ID NO: 11)
FVNQHLCGSHLVEALYLVCGERGFFYTDKX₃₁X₃₀,
(SEQ ID NO: 12)
FVNQHLCGSHLVEALYLVCGERGFFYTKPX₃₁X₃₀,
(SEQ ID NO: 13)
FVNQHLCGSHLVEALYLVCGERGFFYTPKX₃₁X₃₀,
(SEQ ID NO: 14)
FVNQHLCGSHLVEALYLVCGERGFFYTDKX₃₁X₃₂X₃₀,
(SEQ ID NO: 15)
FVNQHLCGSHLVEALYLVCGERGFFYTKPX₃₁X₃₂X₃₀
and
(SEQ ID NO: 16)
FVNQHLCGSHLVEALYLVCGERGFFYTPKX₃₁X₃₂X₃₀,

wherein
X₃₁ and X₃₂ are independently any amino acid; and
X₃₀ is a diol bearing amino acid derivative, optionally threoninol.

In accordance with embodiment 15 an insulin analogue of any one of embodiments 1-14 is provided wherein the A chain is a polypeptide selected from the group consisting of

(SEQ ID NO: 17)
R-GIVEQCCTSICSLYQLENYCN;
and
(SEQ ID NO: 18)
R-GIVEQCCHSICSLYQLENYCN,

wherein
R is

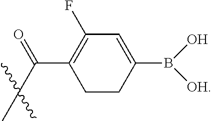 [SMILES: *C(=O)C1=C(F)C=C(B(O)O)CC1]

In accordance with embodiment 16 a method of preparing an analogue of any one of Embodiments 1-15 is provided by means of trypsin-mediated semi-synthesis wherein (a) any optional A-chain modification (i.e., by a monomeric glucose-binding moiety) is introduced within a des-octapeptide[B23-B30] fragment of insulin or insulin analogue and (b) the diol-containing B-chain modification is introduced within a synthetic peptide of length 5-10 amino-acid residues whose N-terminal residue is Glycine and which upon modification contains no tryptic cleavage site.

In accordance with embodiment 17 the method of embodiment 16 is provided wherein the des-octapeptide[B23-B30] fragment of insulin or an insulin analogue is obtained by trypsin digestion of a parent insulin or insulin analogue.

In accordance with embodiment 18 the method of embodiment 16 is provided wherein the des-octapeptide[B23-B30] fragment of insulin or insulin analogue is obtained by trypsin digestion of a single-chain polypeptide (such as proinsulin, a proinsulin analogue or a corresponding mini-proinsulin containing a foreshortened or absent C domain) as expressed in Escherichia coli, Saccharomyces cerevisiae, Pichia pastoris or other microbial system for the recombinant expression of proteins.

In accordance with embodiment 19 the method of embodiment 16 is provided wherein the des-octapeptide[B23-B30] fragment of insulin or insulin analogue is obtained by trypsin digestion of single-chain polypeptide (such as proinsulin, a proinsulin analogue or a corresponding mini-proinsulin containing a foreshortened or absent C domain) as prepared by solid-phase chemical peptide synthesis, optionally including native fragment-ligation steps.

In accordance with embodiment 20, a method of treating a diabetic patient is provided wherein the patient is administered a physiologically effective amount of an insulin analogue of any one of embodiments 1-15, or a physiologically acceptable salt thereof via any standard route of administration.

### Example 1

### A Fructose-Responsive Insulin

A fructose-responsive insulin FRI scheme was prepared as proof of principle that the main-chain directed diols in the B chain can be used to prepare glucose responsive insulin analogs.

A switchable insulin analog (designated FRI; fructose-responsive insulin) contains meta-fluoro-PBA* (meta-fPBA or m-fPBA) as a diol sensor linked to the α-amino group of Gly^{A1} and an aromatic diol (3,4-dihydroxybenzoic acid; DHBA) attached to the e-amino group of Lys^{B28} of insulin lispro. Although fructose and glucose each contain diols, the sensor preferentially binds to aligned 1,2-diol groups as found in β-D-fructofuranose and α-D-glucofuranose. Affinity of meta-fPBA is higher for fructose than glucose due to salient differences in respective conformational; binding is covalent but reversible. To compensate for impairment of IR-binding affinity generally associated with N-linked adducts at Gly^{A1}, Thr^{A8} was substituted by His a favorable substitution found in avian insulins. Control analogs were provided by 1) insulin KP, 2) a KP derivative containing an A1-linked meta-fPBA but not the B28 diol (diol-free control; DFC), and 3) a peptide bond between Lys^{B28} and Gly^{A1} in a des-[B29, B30] template. The latter [a covalent “closed” state] was inactive.

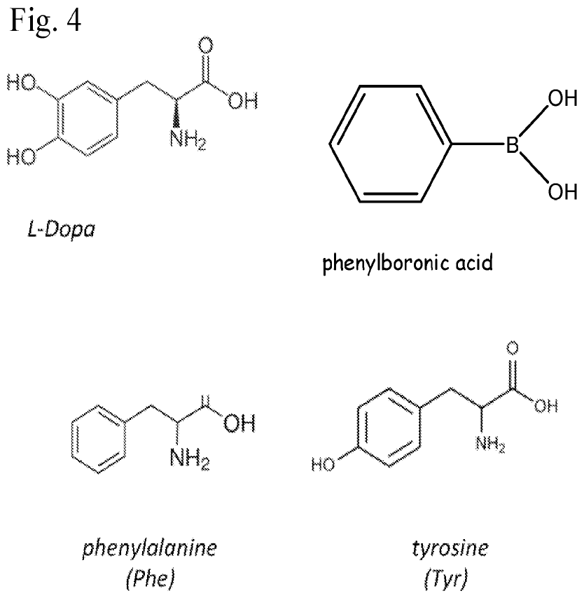

Western Blot Assays Demonstrated Fructose-Dependent Signaling. Structural studies suggest that insulin's hinge-opening at a dimer-related αCT/L1 interface is coupled to closure of IR ectodomain legs, leading to TK-mediated trans-phosphorylation and receptor activation. Signal propagation was probed via a cytoplasmic kinase cascade and changes in metabolic gene expression in HepG2 cells. Control studies indicated that addition of 0 to 100 mM fructose or glucose did not trigger changes in signaling outputs. An overview of IR autophosphorylation (probed by anti-pTyr IR antibodies) and downstream phosphorylation of Ser-Thr protein kinase AKT (protein kinase B; ratio p-AKT/AKT), forkhead transcription factor 1 (p-FOXO1/FOXO1), and glycogen synthase kinase-3 (p-GSK-3/GSK-3) at a single hormone dose (50 nM) was provided by Western blot (WB; FIG. 4 D-F). In each case, WBs demonstrated fructose-dependent signaling by FRI and fructose-independent signaling by KP and DFC. The activity of FRI in the absence of fructose is low.

Plate Assays Demonstrated Ligand-Selective Signaling. Quantitative dose-dependent and ligand-selective IR autophosphorylation were evaluated in a 96-well plate assay (FIG. 5A). FRI triggered a robust signal on addition of 50 mM fructose whereas baseline activity in the absence of fructose was low. As expected, KP and DFC exhibited high signaling activity in the presence or absence of fructose, respectively). Ligand-dependent activation of FRI is specific to fructose as addition of 50 mM glucose did not influence its activity (nor the activities of KP and DFC). These data indicate that in 50 mM fructose FRI is almost as active as KP.

PCR Assays Demonstrated Ligand-Selective Metabolic Gene Regulation. Insulin-signaling in hepatocytes extends to metabolic transcriptional regulation as recapitulated in HepG2 cells. At hypoglycemic conditions, the cells exhibited stronger gluconeogenesis-related responses following insulin stimulation than at hyperglycemic conditions. In this protocol, FRI, when activated by fructose, regulated downstream expression of the gene encoding phosphoenolpyruvate carboxykinase (PEPCK; a marker for hormonal control of gluconeogenesis). Under normoglycemic conditions, FRI, when activated by fructose, regulated the genes encoding carbohydrate-response-element and sterol response-element binding proteins (ChREBP and SREBP; markers for hormonal control of lipid biosynthesis). No fructose dependence was observed in control studies of KP and DFC; no effects were observed on addition of glucose instead of fructose. Control studies were undertaken in the absence of insulin analogs to assess potential confounding changes in metabolic gene expression on addition of 0 to 100 mM fructose or 0 to 100 mM glucose. No significant effects were observed in either case, indicating that the present short-term fructose exposure (to activate FRI) is unassociated with the transcriptional signature of longer-term exposure.

Ligand-Binding to FRI Affects Protein Structure. Far-UV circular dichroism (CD) spectra of FRI and DFC are indistinguishable from parent analog KP (FIG. 7A), indicating that secondary structure is not affected by the modifications at A1 and B28. Difference CD spectra calculated on addition of 100 mM fructose or glucose were in each case featureless. High-resolution NMR spectroscopy [as enabled by the monomeric KP template] corroborate essential elements of the intended fructose-selective switch

¹⁹F-NMR spectra monitored fructose sensor. The fluorine atom in meta-fPBA provided an NMR-active nucleus. Addition of 0 to 100 mM fructose led to an upfield change in 19F-NMR chemical shift in slow exchange on the NMR time scale. This upfield shift presumably reflects displacement of an aromatic diol by a nonaromatic ligand. No change in FRI ¹⁹F chemical shift was observed on addition of glucose. Although an analogous ¹⁹F resonance was observed in the NMR spectrum of DFC, its chemical shift did not change on addition of glucose or fructose. Interestingly, a broadened ¹⁹F signal was observed in ligand-free DFC, probably due to conformational exchange or self-association; this signal sharpened on addition of ligand (fructose or glucose).

Dual ¹⁹F- and ¹H NMR-monitored titration and natural-abundance ¹H-¹³C heteronuclear single quantum coherence (HSQC) spectra provided further evidence of a specific interaction between FRI and fructose.

¹H-¹³C 2D HSQC spectra monitored “closed” conformation of ligand-free FRI. One-dimensional (1D)¹H and ¹H-¹³C HSQC spectra of DFC were similar to those of parent analog KP, excepting methyl resonances of IleA2 and ValA3 (adjacent to the Gly^{A1}-attached meta-fPBA). Patterns of ¹H-¹³C chemical shifts of FRI and DFC were also similar. Those NMR features provided evidence that FRI and DFC retain a native-like structure. However, in FRI, the resonances of Ile^{A2}, Val^{A3}, Leu^{B11} Val^{B12} and Leu^{B15} exhibited larger chemical shift differences (relative to KP) than in DFC. These findings suggest that FRI exhibits a local change in conformation and/or dynamics, presumably due to the intended DHBA/meta-fPBA tether. We envision that constraining the C-terminal B-chain segment alters aromatic ring currents affecting the central B-chain α-helix (via Tyr^{B26}Leu^{B11}, Tyr^{B26}-Val^{B12}, and Phe^{B24}-Leu^{B15} packing) and N-terminal A-chain helix (via native-like TyrB26-IleA2 and Tyr^{B26}-Val^{A3} packing).

Aromatic ¹H-¹³C two-dimensional (2D) HSQC spectra monitor hinge-opening. ¹H-¹³C HSQC spectra provide probes of aromatic resonances in FRI's DHBA/meta-fPBA adducts in the absence of fructose and in the presence of 100 mM fructose. Significant chemical shift changes in both ¹H/¹³C dimensions were observed. Resonance assignments were corroborated by model studies of meta-fPBA- and DHBA-modified peptides. DHBA chemical shifts in fructose-free FRI are similar to those in the complex of model peptides, whereas such chemical shifts in fructose bound FRI are similar to that of free DHBA-modified octapeptide. In addition, methyl resonances sensitive to addition of fructose exhibited a trend toward corresponding chemical shifts observed in spectra of insulin lispro and ligand-free DFC. Together, these NMR features provide evidence that in FRI the Lys^{B28}-attached DHBA binds Gly^{A1}-linked meta-fPBA in absence of fructose, but this tether is displaceable by fructose.

Methyl ¹H-¹³C 2D HSQC spectra monitor protein core. Aliphatic ¹H-¹³C spectra reflect tertiary structure as probed by upfield-shifted methyl resonances. Changes in cross-peak chemical shifts were observed in FRI on overlay of spectra acquired in the absence of an added monosaccharide or on addition of 100 mM fructose. Fructose-binding accentuated upfield ¹H secondary shifts with smaller changes in ¹³C chemical shifts. These changes presumably reflect altered aromatic ring currents within insulin's core. Control studies of DFC suggested that such chemical shift changes require the interchain DHBA/meta-fPBA tether; in these spectra, changes were restricted to IleA2 immediately adjoining the sensor. Addition of 50 mM glucose caused essentially no changes in ¹H-¹³C fingerprints of FRI or DFC in accordance with the fructose selectivity of meta-fPBA.

### DISCUSSION

Engineering of a ligand-regulated switch within a protein requires 1) a ligand-binding element and 2) a mechanism-coupling ligand-binding to a functional step. The present application to insulin exploited the hinge-opening mechanism through which the native hormone interacts with its receptor. Coupling between IR-binding and ligand sensing was provided by an internal interchain tether displaced by the ligand (fructose). Our results provide evidence that hinge opening is required for hormone-triggered receptor autophosphorylation and downstream signaling.

### Engineered Tethers in Proteins.

By analogy to engineered disulfide bridges as reducible probes of protein function, we imagined a ligand-cleavable tether between insulin's A and B chains as a redox-independent switch. This design, making ligand-dependent hinge opening possible, stands in contrast to classical ligand-binding motifs in proteins associated with stabilization of structure. Zn fingers and other Zn-binding motifs, for example, generally exhibit metal ion-dependent peptide folding. Analogous metal ion-coupled folding of RNA underlies the function of riboswitches, control motifs in untranslated messenger RNA (mRNA) regions. Insulin self-assembly itself is stabilized by Zn2+ coordination, whereas the structure of each protomer within the T6 (2-Zn) hexamer is similar to that of the native monomer. Binding of phenolic ligands to this hexamer triggers an allosteric transition, leading to the more-stable R6 state. Containing an extended α-helix, the latter is preferred for pharmaceutical formulations as its greater stability extends shelf life. The present fructose-cleavage interchain tether in FRI provides a contrasting example of ligand-driven loss of structure or stability.

Ligand-induced destabilization of structure has a long history of investigation in relation to glucose-responsive polymers, such as hydrogels designed to swell and release insulin on an increase in local glucose concentration. A well-characterized embodiment is provided by polymer matrices embedded with glucose oxidase and insulin. When the ambient glucose concentration is high, its enzymatic conversion to gluconic acid (in presence of oxygen) causes a reduction in pH, in turn swelling the matrix and enabling insulin release. This “smart” materials approach to engineering a glucose-responsive subcutaneous depot addresses a long-sought but unmet medical need: how to reduce the risk of hypoglycemia in patients receiving insulin replacement therapy. Concerns related to hypoglycemia and its sequelae can limit glycemic targets in Type 1 and long-standing Type 2 DM.

The present monosaccharide-dependent disruption of an interchain tether in FRI extends to the nanoscale the goals of mesoscale glucose-responsive materials engineering. Its molecular design provides proof of principle for a minimal “smart” insulin nanotechnology in the absence of a polymer matrix and with mechanism unrelated to prior proposed unimolecular GRIs. Whereas the fructose-free tethered state would resemble chemically crosslinked or single-chain insulin analogs—long known to exhibit low activities—the fructose-bound open state is competent to bind IR via Site-1-associated detachment of the B24 to B30 segment from the α-helical core of the hormone. We anticipate that replacement of a PBA-based fructose sensor by a bona-fide glucose sensor would provide a Site-1-based GRI of potential clinical utility. This scheme would provide a reversible conformational constraint regulating hormonal activity through changing metabolic conditions. Whereas the selectivity of PBA for fructose is in accordance with the conformational properties of monosaccharides, other types of monosaccharide-recognition elements have been described that recognize the distinctive arrangement of hydroxyl groups well populated among glucose isomers.

What is claimed is:

1. An insulin analogue consisting of an A chain modified by a glucose-binding element at or near its N terminus and a variant B chain comprising a diol group at the C terminus of the B chain such that the polypeptide chain ends with a hydroxyl group rather than with a carboxylate group.

2. An insulin analogue of claim 1 wherein the A chain contains a substitution at position A8 that enhances affinity of the insulin analogue for the insulin receptor, optionally wherein the substitution at position A8 is histidine.

3. An insulin analogue of claim 1 wherein the A chain contains a substitution at position A8 or position A14 that enhances thermodynamic stability of the insulin analogue for the insulin receptor, optionally wherein the substitution at position A8 or A14 is independently selected from the group consisting of His^{A8}, Lys^{AB}, Arg^{A8}, and Glu^{A8}.

4. An insulin analogue of claim 1 wherein the A chain contains a substitution at position A21 that protects the insulin analogue from chemical degradation.

5. An insulin analogue of claim 2 wherein said diol group at the C terminus of the B chain is an aliphatic (1, 2) diol.

6. An insulin analogue of claim 2 wherein said diol group at the C terminus of the B chain is an aliphatic (1, 3) diol.

7. An insulin analogue of claim 1 further comprising a modified amino acid at a position 1, 2, 3, or 4 residues N-terminal to the C-terminal amino acid, wherein said modified amino acid is an L or D amino acid comprising a side-chain diol.

8. An insulin analogue of claim 7 wherein the modified amino acid is thiol-containing L or D amino acid.

9. An insulin analogue of claim 2 further comprising an L Dopa at position B26 or an L or D Dopa located at 1-3 residues N-terminal to the C-terminal amino acid.

10. An insulin analogue of claim 1 where said B chain is a truncated B chain lacking residue B30, residues B29-B30, residues B28-B30, residues B27-B30 or residues B26-B30, with a diol group located at the C terminus of the truncated B chain.

11. An insulin analogue of claim 1 where said B chain is extended by one or two amino acids with a diol group located at the C terminus of the extended B chain.

12. An insulin analogue of claim 1 wherein the B chain is a polypeptide selected from the group consisting of
(SEQ ID NO: 1)
FVKQHLCGSHLVEALYLVCGERGFFYTEKX₃₀,
(SEQ ID NO: 2)
FVNQHLCGSHLVEALYLVCGERGFFYTDKX₃₀,
(SEQ ID NO: 3)
FVNQHLCGSHLVEALYLVCGERGFFYTKPX₃₀,
(SEQ ID NO: 4)
FVNQHLCGSHLVEALYLVCGERGFFYTPKX₃₀
(SEQ ID NO: 5)
FVNQHLCGSHLVEALYLVCGERGFFYTKX₃₀,
(SEQ ID NO: 6)
FVNQHLCGSHLVEALYLVCGERGFFYTPX₂₉X₃₀,
(SEQ ID NO: 7)
FVNQHLCGSHLVEALYLVCGERGFFYTPX₃₀
(SEQ ID NO: 8)
FVNQHLCGSHLVEALYLVCGERGFFYTX₂₉X₃₀
(SEQ ID NO: 9)
FVNQHLCGSHLVEALYLVCGERGFFYTX₃₀
and
(SEQ ID NO: 10)
FVNQHLCGSHLVEALYLVCGERGFFYX₃₀,
wherein
X₂₉ is ornithine; and
X₃₀ is a diol bearing amino acid derivative, optionally threoninol.

13. An insulin analogue of claim 1 wherein the B chain is a polypeptide selected from the group consisting of
(SEQ ID NO: 37)
FVNQHLCGSHLVEALYLVCGERGFF[Sar][APD],
(SEQ ID NO: 38)
FVNQHLCGSHLVEALYLVCGERGFF[dA][APD],
and
(SEQ ID NO: 39)
FVNQHLCGSHLVEALYLVCGERGFFYG[APD],
wherein APD is 3-amino-1,2-propandiol.

14. An insulin analogue of claim 1 wherein the B chain is a polypeptide selected from the group consisting of
(SEQ ID NO: 11)
FVNQHLCGSHLVEALYLVCGERGFFYTDKX₃₁X₃₀,
(SEQ ID NO: 12)
FVNQHLCGSHLVEALYLVCGERGFFYTKPX₃₁X₃₀,
(SEQ ID NO: 13)
FVNQHLCGSHLVEALYLVCGERGFFYTPKX₃₁X₃₀,
(SEQ ID NO: 14)
FVNQHLCGSHLVEALYLVCGERGFFYTDKX₃₁X₃₂X₃₀,
(SEQ ID NO: 15)
FVNQHLCGSHLVEALYLVCGERGFFYTKPX₃₁X₃₂X₃₀
and
(SEQ ID NO: 16)
FVNQHLCGSHLVEALYLVCGERGFFYTPKX₃₁X₃₂X₃₀,
wherein
X₃₁ and X₃₂ are independently any amino acid; and
X₃₀ is a diol bearing amino acid derivative, optionally threoninol.

15. An insulin analogue of claim 12 or 13 wherein the A chain is a polypeptide selected from the group consisting of
(SEQ ID NO: 17)
R-GIVEQCCTSICSLYQLENYCN;
and
(SEQ ID NO: 18)
R-GIVEQCCHSICSLYQLENYCN,
wherein
R is

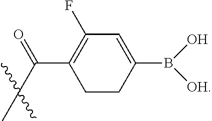 [SMILES: *C(=O)C1=C(F)C=C(B(O)O)CC1]

16. A method of preparing an analogue of any one of claims 1-15 by means of trypsin-mediated semi-synthesis wherein (a) any optional A-chain modification (i.e., by a monomeric glucose-binding moiety) is introduced within a des-octapeptide[B23-B30] fragment of insulin or insulin analogue and (b) the diol-containing B-chain modification is introduced within a synthetic peptide of length 5-10 amino-acid residues whose N-terminal residue is Glycine and which upon modification contains no tryptic cleavage site.

17. The method of claim 16 wherein the des-octapeptide[B23-B30] fragment of insulin or an insulin analogue is obtained by trypsin digestion of a parent insulin or insulin analogue.

18. A method of claim 16 wherein the des-octapeptide[B23-B30] fragment of insulin or insulin analogue is obtained by trypsin digestion of a single-chain polypeptide (such as proinsulin, a proinsulin analogue or a corresponding mini-proinsulin containing a foreshortened or absent C domain) as expressed in Escherichia coli, Saccharomyces cerevisiae, Pichia pastoris or other microbial system for the recombinant expression of proteins.

19. The method of claim 16 wherein the des-octapeptide[B23-B30] fragment of insulin or insulin analogue is obtained by trypsin digestion of single-chain polypeptide (such as proinsulin, a proinsulin analogue or a corresponding mini-proinsulin containing a foreshortened or absent C domain) as prepared by solid-phase chemical peptide synthesis, optionally including native fragment-ligation steps.

20. A method of treating a diabetic patient comprising administering a physiologically effective amount of an insulin analogue of any one of claims 1-15, or a physiologically acceptable salt thereof to the patient.


SEQUENCE LISTING (40 sequences)

SEQ ID NO: 1 - protein, 30 aa, Homo sapiens
  MISC_FEATURE (30)..(30): Xaa at position 30 is a diol bearing amino acid derivative
FVKQHLCGSHLVEALYLVCGERGFFYTEKX

SEQ ID NO: 2 - protein, 30 aa, Homo sapiens
  MISC_FEATURE (30)..(30): Xaa at position 30 is a diol bearing amino acid derivative
FVNQHLCGSHLVEALYLVCGERGFFYTDKX

SEQ ID NO: 3 - protein, 30 aa, Homo sapiens
  MISC_FEATURE (30)..(30): Xaa at position 30 is a diol bearing amino acid derivative
FVNQHLCGSHLVEALYLVCGERGFFYTKPX

SEQ ID NO: 4 - protein, 30 aa, Homo sapiens
  MISC_FEATURE (30)..(30): Xaa at position 30 is a diol bearing amino acid derivative
FVNQHLCGSHLVEALYLVCGERGFFYTPKX

SEQ ID NO: 5 - protein, 29 aa, Homo sapiens
  misc_feature (29)..(29): Xaa can be any naturally occurring amino acid
  MISC_FEATURE (30)..(30): Xaa at position 30 is a diol bearing amino acid derivative
FVNQHLCGSHLVEALYLVCGERGFFYTKX

SEQ ID NO: 6 - protein, 30 aa, Homo sapiens
  MISC_FEATURE (29)..(29): Xaa at position 29 is ornithine
  MISC_FEATURE (30)..(30): Xaa at position 30 is a diol bearing amino acid derivative
FVNQHLCGSHLVEALYLVCGERGFFYTPXX

SEQ ID NO: 7 - protein, 29 aa, Homo sapiens
  MISC_FEATURE (29)..(29): Xaa at position 29 is a diol bearing amino acid derivative
FVNQHLCGSHLVEALYLVCGERGFFYTPX

SEQ ID NO: 8 - protein, 29 aa, Homo sapiens
  MISC_FEATURE (28)..(28): Xaa at position 28 is ornithine
  MISC_FEATURE (29)..(29): Xaa at position 29 is a diol bearing amino acid derivative
FVNQHLCGSHLVEALYLVCGERGFFYTXX

SEQ ID NO: 9 - protein, 28 aa, Homo sapiens
  MISC_FEATURE (28)..(28): Xaa at position 28 is a diol bearing amino acid derivative
FVNQHLCGSHLVEALYLVCGERGFFYTX

SEQ ID NO: 10 - protein, 27 aa, Homo sapiens
  MISC_FEATURE (27)..(27): Xaa at position 27 is a diol bearing amino acid derivative
FVNQHLCGSHLVEALYLVCGERGFFYX

SEQ ID NO: 11 - protein, 31 aa, Homo sapiens
  misc_feature (30)..(30): Xaa can be any naturally occurring amino acid
  MISC_FEATURE (31)..(31): Xaa at position 31 is a diol bearing amino acid derivative
FVNQHLCGSHLVEALYLVCGERGFFYTDKXX

SEQ ID NO: 12 - protein, 31 aa, Homo sapiens
  misc_feature (30)..(30): Xaa can be any naturally occurring amino acid
  MISC_FEATURE (31)..(31): Xaa at position 31 is a diol bearing amino acid derivative
FVNQHLCGSHLVEALYLVCGERGFFYTKPXX

SEQ ID NO: 13 - protein, 31 aa, Homo sapiens
  misc_feature (30)..(30): Xaa can be any naturally occurring amino acid
  MISC_FEATURE (31)..(31): Xaa at position 31 is a diol bearing amino acid derivative
FVNQHLCGSHLVEALYLVCGERGFFYTPKXX

SEQ ID NO: 14 - protein, 32 aa, Homo sapiens
  misc_feature (30)..(31): Xaa can be any naturally occurring amino acid
  MISC_FEATURE (32)..(32): Xaa at position 32 is a diol bearing amino acid derivative
FVNQHLCGSHLVEALYLVCGERGFFYTDKXXX

SEQ ID NO: 15 - protein, 32 aa, Homo sapiens
  misc_feature (30)..(31): Xaa can be any naturally occurring amino acid
  MISC_FEATURE (32)..(32): Xaa at position 32 is a diol bearing amino acid derivative
FVNQHLCGSHLVEALYLVCGERGFFYTKPXXX

SEQ ID NO: 16 - protein, 32 aa, Homo sapiens
  misc_feature (30)..(31): Xaa can be any naturally occurring amino acid
  MISC_FEATURE (32)..(32): Xaa at position 32 is a diol bearing amino acid derivative
FVNQHLCGSHLVEALYLVCGERGFFYTPKXXX

SEQ ID NO: 17 - protein, 21 aa, Homo sapiens
GIVEQCCTSICSLYQLENYCN

SEQ ID NO: 18 - protein, 21 aa, Homo sapiens
GIVEQCCHSICSLYQLENYCN

SEQ ID NO: 19 - protein, 8 aa, Homo sapiens
  MISC_FEATURE (8)..(8): Xaa at position 8 is Threoninol
GFFYTKPX

SEQ ID NO: 20 - protein, 7 aa, Homo sapiens
  MISC_FEATURE (7)..(7): Xaa at position 7 is Threoninol
GFFYTKX

SEQ ID NO: 21 - protein, 6 aa, Homo sapiens
  MISC_FEATURE (6)..(6): Xaa at position 6 is Threoninol
GFFYTX

SEQ ID NO: 22 - protein, 5 aa, Homo sapiens
  MISC_FEATURE (5)..(5): Xaa at position 5 is Threoninol
GFFYX

SEQ ID NO: 23 - protein, 7 aa, Homo sapiens
  MISC_FEATURE (6)..(6): Xaa at position 6 is Ornithine
  MISC_FEATURE (7)..(7): Xaa at position 7 is Threoninol
GFFYTXX

SEQ ID NO: 24 - protein, 7 aa, Homo sapiens
  MISC_FEATURE (7)..(7): Xaa at position 7 is Threoninol
GFFYTPX

SEQ ID NO: 25 - protein, 8 aa, Homo sapiens
  MISC_FEATURE (7)..(7): Xaa at position 7 is Ornithine
  MISC_FEATURE (8)..(8): Xaa at position 8 is Threoninol
GFFYTPXX

SEQ ID NO: 26 - protein, 5 aa, Homo sapiens
  MISC_FEATURE (5)..(5): Xaa at position 5 is ADP
GFFYX

SEQ ID NO: 27 - protein, 4 aa, Homo sapiens
  MISC_FEATURE (4)..(4): Xaa at position 4 is tBu tyrosine
GFFX

SEQ ID NO: 28 - protein, 6 aa, Homo sapiens
  MISC_FEATURE (6)..(6): Xaa at position 6 is ADP
GFFYGX

SEQ ID NO: 29 - protein, 5 aa, Homo sapiens
  MISC_FEATURE (4)..(4): Xaa at position 4 is tBu tyrosine
GFFXG

SEQ ID NO: 30 - protein, 6 aa, Homo sapiens
  MISC_FEATURE (6)..(6): Xaa at position 6 is ADP
GFFYTX

SEQ ID NO: 31 - protein, 5 aa, Homo sapiens
  MISC_FEATURE (4)..(4): Xaa at position 4 is tBu tyrosine
  MISC_FEATURE (5)..(5): Xaa at position 5 is tBu threonine
GFFXX

SEQ ID NO: 32 - protein, 7 aa, Homo sapiens
  MISC_FEATURE (7)..(7): Xaa at position 7 is ADP
GFFYTGX

SEQ ID NO: 33 - protein, 6 aa, Homo sapiens
  MISC_FEATURE (4)..(4): Xaa at position 4 is tBu tyrosine
  MISC_FEATURE (5)..(5): Xaa at position 5 is tBu threonine
GFFXXG

SEQ ID NO: 34 - protein, 5 aa, Homo sapiens
  MISC_FEATURE (4)..(4): Xaa at position 4 is sarcosine
  MISC_FEATURE (5)..(5): Xaa at position 5 is ADP
GFFXX

SEQ ID NO: 35 - protein, 4 aa, Homo sapiens
  MISC_FEATURE (4)..(4): Xaa at position 4 is sarcosine
GFFX

SEQ ID NO: 36 - protein, 5 aa, Homo sapiens
  MISC_FEATURE (4)..(4): Xaa at position 4 is D-alanine
  MISC_FEATURE (5)..(5): Xaa at position 5 is ADP
GFFXX

SEQ ID NO: 37 - protein, 27 aa, Homo sapiens
  MISC_FEATURE (26)..(26): Xaa at position 26 is sarcosine
  MISC_FEATURE (27)..(27): Xaa at position 27 is ADP
FVNQHLCGSHLVEALYLVCGERGFFXX

SEQ ID NO: 38 - protein, 27 aa, Homo sapiens
  MISC_FEATURE (26)..(26): Xaa at position 26 is dAla
  MISC_FEATURE (27)..(27): Xaa at position 27 is ADP
FVNQHLCGSHLVEALYLVCGERGFFXX

SEQ ID NO: 39 - protein, 28 aa, Homo sapiens
  MISC_FEATURE (28)..(28): Xaa at position 28 is ADP
FVNQHLCGSHLVEALYLVCGERGFFYGX

SEQ ID NO: 40 - protein, 9 aa, Homo sapiens
  MISC_FEATURE (9)..(9): Xaa at position 9 is Threoninol
GYYFTTKPX